(19) United States
(12) Patent Application Publication
(10) Pub. No.: US 2009/0247887 A1
(43) Pub. Date: Oct. 1, 2009

(54) FLOW MEASUREMENT IN GRAFTS

(76) Inventors: David Hull, West Hartford, CT (US); Charles W. Cannon, JR., Jersey Shore, PA (US)

Correspondence Address: CANTOR COLBURN, LLP, 20 Church Street, 22nd Floor, Hartford, CT, 06103, US

(21) Appl. No.: 12/413,715

(22) Filed: Mar. 30, 2009

Related U.S. Application Data
(60) Provisional application No. 61/040,294, filed on Mar. 28, 2008.

(51) Int. Cl. A61B 5/02

(52) U.S. Cl. 600/505

(57) ABSTRACT

A system for conducting flow measurements in grafts is provided, comprising a graft that further includes a monitor placed within or on the graft, wherein the monitor measures flow. In exemplary embodiments, the monitor is an embedded monitor that is placed within or on a vascular bypass graft that is commonly used for arterial and venous bypass or for hemo-dialysis access, ultrafiltration, or phoresis. The exemplary monitoring system may measure flow by electrical impedance, electronic and/or magnetic flux. An external monitor may also be intermittently applied. Alternately, a continuous reporting can be performed by telepathic signals from the graft transmitter.

### CROSS REFERENCE TO RELATED APPLICATIONS

The present application claims priority to U.S. Provisional Patent Application Ser. No. 61/040,294, filed Mar. 28, 2008, the entire contents of which are specifically incorporated by reference herein.

### BACKGROUND

Vascular disease and renal failure are illnesses that continue to place a great demand on the health system. In order to address vascular disease and renal failure, vascular bypass and vascular access processes are commonly performed.

Once placed successfully (through a number of physiologic mechanisms and surgical procedures), bypass grafts are at risk for failure from a number of factors. Some factors include progression or development of vascular disease, alteration or production of blood elements leading to clotting and/or increased viscosity, and unique changes related to graft placement that cause fibrosis such as intimal hyperplasia, etc.

Currently there is no known way to accurately measure, in real time, graft and vessel flow rates. Current measurements of flow rates are actually calculated using assumptions that are often inaccurate. The end result is too often graft failure, which may lead to tissue and limb injury and or limb loss, potential death and emergent surgical and or interventional radiologic procedures. In hemo-dialysis patients, graft failures lead to inadequate dialysis, risk for other temporary devices such as catheter placement for interim hemodialysis, hospital admissions, deceased longevity of the access needing surgical or interventional radiologic procedures and or revision and potential new access placement in patients with finite sites for graft placement.

What is needed in the care of these grafts and bypasses is a more effective system for conducting flow measurements.

### SUMMARY

The above described and other deficiencies of the prior bypasses and grafts are overcome and alleviated by the present system for conducting flow measurements in grafts, comprising a graft that further includes a monitor placed within or on the graft, wherein the monitor measures flow.

In exemplary embodiments, the monitor is an embedded device which is placed within or on a vascular bypass graft that is commonly used for arterial and venous bypass or for hemo-dialysis access, ultrafiltration, or phoresis. The exemplary monitoring system may measure flow by electrical impedance, electronic and/or magnetic flux.

An external monitor may also be intermittently applied. Alternately, a continuous reporting can be performed by telepathic signals from the graft transmitter.

Once flow rates are determined, alteration of flow will allow prompt medical surveillance for diagnostic and or therapeutic intervention. This will prevent unrecognized alterations in flow from vascular abnormalities or disease progression or development (i.e. atherosclerosis, arteriolosclerosis, intimal hyperplasia, plaque fractures, compression from hemotomas, edema, trauma, etc).

The vascular flow monitor will allow the patients bypass to be monitored allowing early diagnosis of alterations in flow rates that will allow elective diagnostic and therapeutic interventions.

### BRIEF DESCRIPTION OF THE DRAWINGS

Referring now to the drawings, wherein like elements are numbered alike in the following FIGURE:

FIG. 1 is an exemplary illustration of a graft including a flow monitor attached thereto;

FIG. 2 is an exemplary illustration of a graft installed in a patient, wherein the graft communicates to an external reader via wireless transmission; and

FIG. 3 is an exemplary illustration of an installed graft communicating with a plurality of detectors connected to a display or a processor and display.

### DETAILED DESCRIPTION OF EXEMPLARY EMBODIMENTS

As is discussed above, the present invention relates to a system for conducting flow measurements in grafts, comprising a graft that further includes a monitor placed within or on the graft, wherein the monitor measures flow.

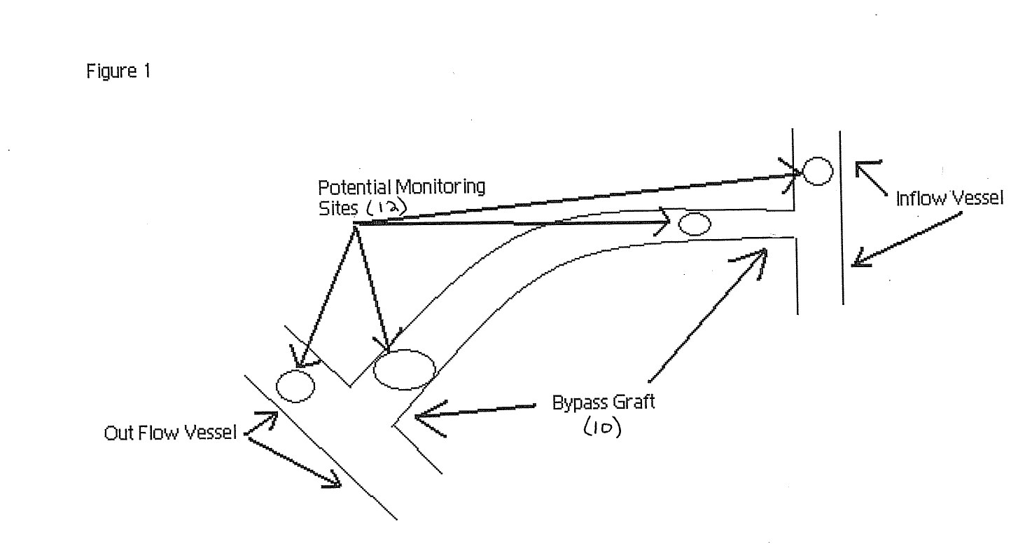

Referring now to FIG. 1, an exemplary vascular bypass graft 10 is illustrated including a flow monitor 12 attached to the vascular graft. While the illustrated monitor is attached to the graft, the monitor may also be embedded or otherwise placed within or on the graft. Exemplary grafts include such that are commonly used for arterial and venous bypass or for hemo-dialysis access, ultrafiltration, or phoresis. The exemplary monitoring system may measure flow by electrical impedance, electronic and/or magnetic flux.

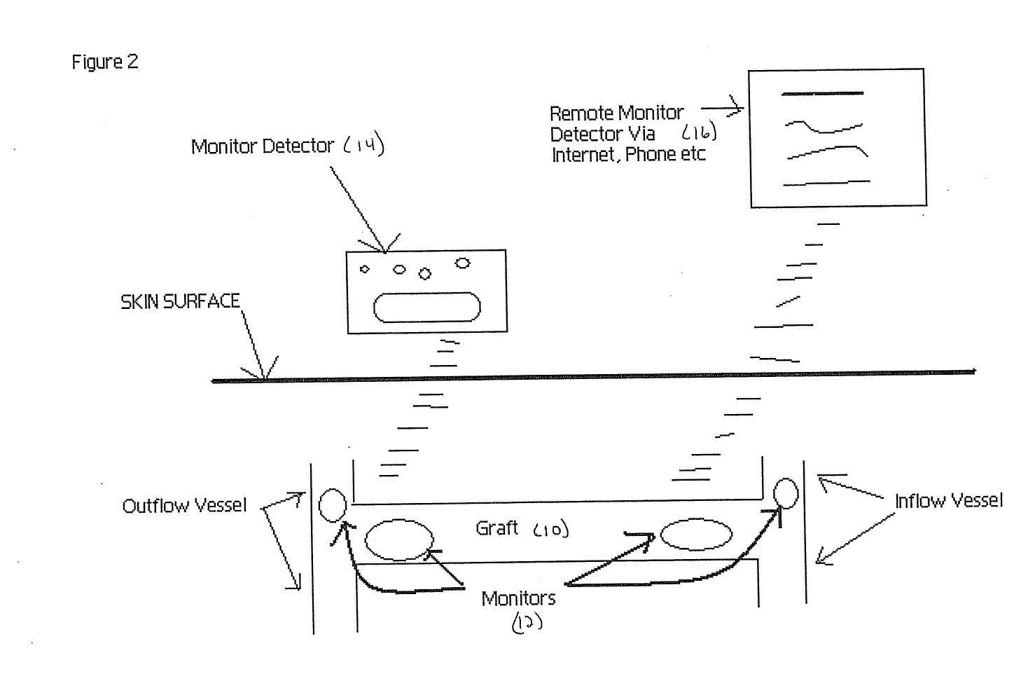

Referring now to FIG. 2, an exemplary external monitor 14 is illustrated as being configured to wirelessly communicate with the graft 10 that is installed within the patient. Communication links may be intermittently applied or a continuous reporting can be performed by telepathic signals from the graft transmitter. Remote monitors 16 may also be used, which monitors are linked to the graft monitors via additional networking mechanisms (e.g., the Internet, telephone phone lines, etc.).

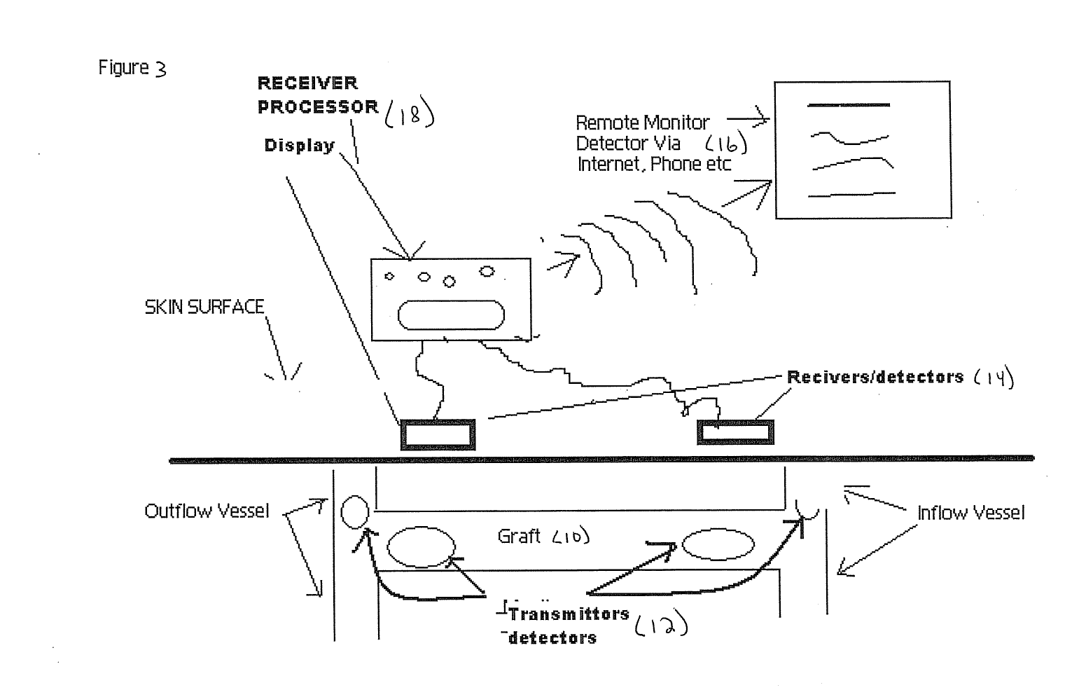

Referring to an exemplary configuration illustrated in FIG. 3, a plurality of detectors 14 may also be used to communicate with the graft flow monitors 12. These detectors may communicate with a central display or receiver processor and display 18, which may also communicate with one or more remote monitors 16.

Once flow rates are determined, alteration of flow will allow prompt medical surveillance for diagnostic and or therapeutic intervention. This will prevent unrecognized alterations in flow from vascular abnormalities or disease progression or development (i.e. atherosclerosis, arteriolosclerosis, intimal hyperplasia, plaque fractures, compression from hemotomas, edema, trauma, etc).

The vascular flow monitor will allow the patients bypass to be monitored allowing early diagnosis of alterations in flow rates that will allow elective diagnostic and therapeutic interventions

It will be apparent to those skilled in the art and science of medicine and surgery that, while exemplary embodiments have been shown and described, various modifications and variations can be made to the system for conducting flow measurement in grafts disclosed herein without departing from the spirit or scope of the invention. Accordingly, it is to be understood that the various embodiments have been described by way of illustration and not limitation.

What is claimed is:

1. A flow monitoring system, comprising:
a synthetic graft or a biologic graft; and
a flow monitoring device that is attached to said graft or embedded in a portion of said graft.

2. A flow monitoring system in accordance with claim 1, wherein the flow monitoring device measures and monitors vascular flow.

3. A flow monitoring system in accordance with claim 1, wherein the flow monitoring device is configured to continuously update an external reading device.

4. A flow monitoring system in accordance with claim 1, wherein the flow monitoring device is configured to intermittently update an external reading device.

5. A flow monitoring system in accordance with claim 1, wherein the flow monitoring device measures flow by electrical impedance, electronic and/or magnetic flux.

6. A flow monitoring system in accordance with claim 1, wherein an external reading device is configured to receive flow measurements from the flow monitoring device via wireless transmission.
